(19) United States
(12) Patent Application Publication
(10) Pub. No.: US 2008/0019936 A1
(43) Pub. Date: Jan. 24, 2008

(54) Multi-Purpose Shave Composition

(76) Inventor: Nikita Wilson, Union, NJ (US)

Correspondence Address: Nikita Wilson, 408 Wallingford Terrace, Union, NJ, 07083, US

(21) Appl. No.: 11/459,490

(22) Filed: Jul. 24, 2006

(51) Int. Cl. A61K 8/73; A61K 8/37

(52) U.S. Cl. 424/70.13; 424/70.22

(57) ABSTRACT

This invention relates to the composition of a multi-purpose shaving composition; designed to work with electric shaving devices and manual razors.

### FIELD OF THE INVENTION

This invention relates to a shave gel or composition that can be used with electric shaving devices and manual razors. The dense lubricious lather enables a close shave with a manual razor and when rinsed off; the conditioning properties of the gel allow the hair to elongate resulting in a closer shave with electric shaving devices.

### BACKGROUND OF THE INVENTION

There are a plethora of electric and manual shaving devices on the market. Along with those devices many shaving gels, lotions, creams and lubrications are sold, some in combination with the shaving apparatus to help assist with the shaving process. In all cases, these shaving formulas serve one purpose to one type of shaving device.

There are many types of people who may use both electric and manual shaving devices. Categorically, those of African descent or those with thick curly hair are more likely to use electric shaving devices, specifically those who suffer from razor bumps or Pseudofolliculitis barbae and would like to receive a closer shave. Those who use manual razors would also benefit from a multipurpose shave gel when they want to switch to an electric solution.

A multipurpose shaving solution is needed that will cater to the varying skin and hair types. A shaving solution that is lubricious enough for manual razors and a preparatory skin and hair conditioner for those who use electric shaving devices is needed.

### DESCRIPTION OF THE RELATED ART

As can be seen by reference to the following U.S. Pat. Nos. 5,262,154; 5,756,081; 5,956,848; 6,096,386; and 6,149,981; the prior art are constructions of either a shave gel or preparatory treatment.

While all of the aforementioned prior art constructions are more than adequate for the basic purpose and function for which they have been specifically designed, none of them serve a multifunctional purpose of a shaving gel and preparatory treatment.

### SUMMARY OF THE INVENTION

The present invention is directed to shave gel compositions comprising from about 0.05% to about 3.0% insoluble cationic conditioner, from about 0.05% to about 5% by weight of a silicone hair conditioning agent, from about 5% to about 60% by weight of a detersive surfactant, from about 0.025% to about 1.5% by weight of selected polyalkylene glycols, preferably polyethylene glycols having from about 1,500 to about 25,000 degrees of ethoxylation, from about 0.25% to about 10% lactylate, from about 0.05% to about 3% water soluble quaternary cellulose derivative and water, and optionally one or more additional materials known for use in conditioning compositions, which compositions provide excellent lubricating and conditioning benefits, and further provide a denser, thicker lather.

### DETAILED DESCRIPTION OF THE INVENTION

The shave gel compositions and corresponding methods of the present invention can comprise, consist of, or consist essentially of the essential elements and limitations of the invention described herein, as well any of the additional ingredients, components, or limitations described herein. All documents referred to herein are incorporated by reference herein in their entirety.

As used herein, “water soluble” refers to any material that is sufficiently soluble in water to form a substantially clear solution to the naked eye at a concentration of 0.1% in water, i.e. distilled or equivalent, at 25.degree. C.

All percentages, parts and ratios are based upon the total weight of the shave gel compositions of the present invention unless otherwise specified.

### Detersive Surfactant

The shave gel compositions of the present invention comprise one or more detersive surfactants selected from the group consisting of anionic surfactant, nonionic surfactant, amphoteric surfactant, zwitterionic surfactants, and mixtures thereof. The shampoo compositions preferably comprise an anionic surfactant. Surfactant concentrations range from about 5% to about 60%, preferably from about 10% to about 55%,

### Anionic Surfactant

The shave gel compositions preferably comprise an anionic surfactant, and preferably at concentrations of from about 5% to about 60%, more preferably from about 10% to about 55%, even more preferably from about 15% to about 50%, and most preferably from about 20% to about 45%, by weight of the composition.

### Amphoteric and Zwitterionic Surfactants

The detersive surfactant of the shaving gel compositions may comprise an amphoteric and/or zwitterionic surfactant. Concentrations of such surfactants will generally range from about 0.5% to about 20%, preferably from about 1% to about 10%, by weight of the shampoo compositions.

Amphoteric surfactants for use in the shaving gel compositions include the derivatives of aliphatic secondary and tertiary amines in which the aliphatic radical is straight or branched and one of the aliphatic substituents contains from about 8 to about 18 carbon atoms and one contains an anionic water solubilizing group, e.g., carboxy, sulfonate, sulfate, phosphate, or phosphonate.

### Nonionic Surfactant

The shaving gel compositions of the present invention may comprise a nonionic surfactant as the detersive surfactant component therein. Nonionic surfactants include those compounds produced by condensation of alkylene oxide groups (hydrophilic in nature) with an organic hydrophobic compound, which may be aliphatic or alkyl aromatic in nature.

Preferred nonionic surfactants for use in the shampoo compositions include the following:

(1) polyethylene oxide condensates of alkyl phenols,

(2) those derived from the condensation of ethylene oxide with the product resulting from the reaction of propylene oxide and ethylene diamine products;

(3) condensation products of aliphatic alcohols having from about 8 to about 18 carbon atoms, in either straight chain or branched chain configuration, with ethylene oxide, e.g.,

(6) long chain dialkyl sulfoxides containing one short chain alkyl or hydroxy alkyl radical of from about 1 to about 3 carbon atoms (usually methyl) and one long hydrophobic chain which include alkyl, alkenyl, hydroxy alkyl, or keto alkyl radicals containing from about 8 to about 20 carbon atoms, from 0 to about 10 ethylene oxide moieties and from 0 to about 1 glyceryl moiety;

(7) alkyl polysaccharide (APS) surfactants (e.g. alkyl polyglycosides), examples of which are described in U.S. Pat. No. 4,565,647, which description is incorporated herein by reference, and which discloses APS surfactants having a hydrophobic group with about 6 to about 30 carbon atoms and polysaccharide (e.g., polyglycoside) as the hydrophilic group; optionally, there can be a polyalkylene-oxide group joining the hydrophobic and hydrophilic moieties; and the allyl group (i.e., the hydrophobic moiety) can be saturated or unsaturated, branched or unbranched, and unsubstituted or substituted (e.g., with hydroxy or cyclic rings); and

(8) polyethylene glycol (PEG) glyceryl fatty esters, such as those of the formula R(O)OCH.sup.2 CH(OH)CH.sup.2 (OCH.sup.2 CH.sup.2).sub.n OH wherein n is from about 5 to about 200, preferably from about 20 to about 100, and R is an aliphatic hydrocarbyl having from about 8 to about 20 carbon atoms.

### Silicone Hair Conditioning Agent

The shave gel compositions of the present invention comprise a silicone hair conditioning agent at concentrations effective to provide hair conditioning benefits. Such concentrations range from about 0.05% to about 10%, preferably from about 0.5% to about 8%, more preferably from about 1.0% to about 5%, most preferably from about 1.5% to about 3%, by weight of the shampoo compositions.

The ethoxylated silicone hair conditioning agents for use in the shave gel compositions are variably soluble in the shave gel compositions depending upon the degree of ethoxylation and are preferably nonvolatile. The degree of ethoxylation can be from about 1 to 200. Cationic silicones are also suitable and included in the scope of this patent.

### Polyalkylene Glycol

The shave gel compositions of the present invention comprise selected polyalkylene glycols in amounts effective to enhance lather performance and enhance spreadability of the shampoo compositions on hair. Effective concentrations of the selected polyethylene glycols range from about 0.025% to about 1.5%, preferably from about 0.05% to about 1%, more preferably from about 0.1% to about 0.5%, by weight of the shave gel compositions.

The polyalkylene glycols suitable for use in the shave gel compositions are characterized by the general formula: ##STR12## wherein R is hydrogen, methyl or mixtures thereof, preferably hydrogen, and n is an integer having an average value of from about 1,500 to about 25,000, preferably from about 2,500 to about 20,000, and more preferably from about 3,500 to about 15,000. When R is hydrogen, these materials are polymers of ethylene oxide, which are also known as polyethylene oxides, polyoxyethylenes, and polyethylene glycols. When R is methyl, these materials are polymers of propylene oxide, which are also known as polypropylene oxides, polyoxypropylenes, and polypropylene glycols. When R is methyl, it is also understood that various positional isomers of the resulting polymers can exist.

Specific examples of suitable polyethylene glycol polymers include PEG-2M wherein R equals hydrogen and n has an average value of about 2,000; PEG-5M wherein R is hydrogen and n has an average value of about 5; PEG-7M wherein R is hydrogen and n has an average value of about 7; PEG-9M wherein R is hydrogen and n has an average value of about 9,000; and PEG-14M wherein R is hydrogen and n has an average value of about 14,000.

Suitable polyaikylene polymers include polypropylene glycois and mixed polyethylene/polypropylene glycols.

It has been found that these polyalkylene glycols, when added to the conditioning shave gel compositions described herein, enhance lather performance in delivering a richer, denser lather feel as well as a lubriciousness that is conducive to a shaving product. Polyethylene glycols, for example, are known for use in improving lather performance in cleansing compositions, Applicants are aware of prior art which teaches the use of these selected polyalkylene glycols in silicone-containing shampoo compositions. However, there is no such data for a shave gel.

It has also been found that these selected polyalkylene glycols, when added to shave gel composition, enhance spreadability and lubriciousness of the shave gel compositions in hair. Enhanced spreading, slip and enhanced conditioning of the shave gel composition provides consumers with a smoother, closer shave.

### Suspending Agent

The shave gel compositions of the present invention comprise a suspending agent at concentrations effective for suspending the silicone hair conditioning agent in dispersed form in the shampoo compositions. Such concentrations range from about 0.1% to about 10%, preferably from about 0.3% to about 5.0%, by weight of the shave gel compositions.

Suitable suspending agents include acyl derivatives, long chain amine oxides, and mixtures thereof, concentrations of which range from about 0.1% to about 5.0%, preferably from about 0.5% to about 3.0%, by weight of the shave gel compositions. These preferred suspending agents include ethylene glycol esters of fatty acids preferably having from about 16 to about 22 carbon atoms. More preferred are the ethylene glycol stearates, both mono and distearate, but particularly the distearate containing less than about 7% of the mono stearate. Other suitable suspending agents include alkanol amides of fatty acids, preferably having from about 16 to about 22 carbon atoms, more preferably about 16 to 18 carbon atoms, preferred examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); glyceryl esters (e.g., glyceryl distearate) and long chain esters of long chain alkanol amides (e.g., stearamide diethanolamide distearate, stearamide monoethanolamide stearate). Long chain acyl derivatives, ethylene glycol esters of long chain carboxylic acids, long chain amine oxides, and alkanol amides of long chain carboxylic acids in addition to the preferred materials listed above may be used as suspending agents. For example, it is contemplated that suspending agents with long chain hydrocarbyls having C.sub.8 −C.sub.22 chains may be used.

Other suitable suspending agents may be used in the shave gel compositions, including those that can impart a gel-like viscosity to the composition, such as water soluble or colloidally water soluble polymers like cellulose ethers (e.g., methylcellulose, hydroxybutyl methylcellulose, hydroxypropylcellulose, hydroxypropyl methylcellulose, hydroxyethyl ethyl cellulose and hydroxyethylcellulose), guar gum, polyvinyl alcohol, polyvinyl pyrrolidone, hydroxypropyl guar gum, starch and starch derivatives, and other thickeners, viscosity modifiers, gelling agents, etc. Mixtures of these materials can also be used.

### Water

The shave gel compositions of the present invention comprise from about 20% to about 94.8%, preferably from about 50% to about 94.8%, more preferably from about 60% to about 85%, by weight of water.

### Optional Hair Conditioning Agents

The shave gel compositions of the present invention may further comprise water soluble cationic polymeric conditioning agents, hydrocarbon conditioning agents, cationic surfactants, and mixtures thereof.

### Cationic Polymer

Optional cationic polymers for use as hair conditioning agents are those having a weight average molecular weight of from about 5,000 to about 10 million, and will generally have cationic, nitrogen-containing moieties such as quaternary ammonium or cationic amino moieties, and mixtures thereof.

Other cationic polymers that can be used include polysaccharide polymers, such as cationic cellulose derivatives, cationic starch derivative, and cationic guar gum derivatives. Other material include quaternary nitrogen-containing cellulose ethers as described in U.S. Pat. No. 3,962,418, and copolymers of etherified cellulose and starch as described in U.S. Pat. No. 3,958,581, which descriptions are incorporated herein by reference.

### Cationic Surfactants

Optional cationic surfactants for use as hair conditioning agents in the shave gel compositions will typically contain quaternary nitrogen moieties. Examples of suitable cationic surfactants are described in following documents, all of which are incorporated by reference herein in their entirety: M.C. Publishing Co., McCutcheon's, Detergents & Emulsifiers, (North American edition 1979); Schwartz, et al., Surface Active Agents, Their Chemistry and Technology, New York: Interscience Publishers, 1949; U.S. Pat. No. 3,155,591; U.S. Pat. No. 3,929,678; U.S. Pat. No. 3,959,461 and U.S. Pat. No. 4,387,090.

### Other Optional Materials

The shave gel compositions of the present invention may comprise one or more optional ingredients to improve or otherwise modify a variety of product characteristics, including aesthetics, stability and use benefits. Many such optional ingredients are known in the art and may be used in the shave gel compositions herein, provided that such ingredients are compatible with the essential ingredients described herein, or do not otherwise unduly impair cleansing or conditioning performance of the shave gel compositions.

Optional materials include foam boosters, preservatives, thickeners, cosurfactants, dyes, perfumes, solvents, styling polymers, anti-static agents, anti-dandruff aids, and pediculocides.

### Method of Use

The shave gel compositions of the present invention are to be use with electric shaving devices or manual razors. An effective amount of the composition for shaving with a manual razor is applied to the area to be shaved, which has preferably been wetted, generally with water, and worked into a lather with either hands or a shaving brush. Such effective amounts generally range from about 1 g to about 10 g, preferably from about 1 to about 5 g. The area is then shaved as per users normal routine For use with electric clippers; an effective amount of the composition for shaving with a manual razor is applied to the area to be shaved, which has preferably been wetted, generally with water, rinsed and blotted dry. Such effective amounts generally range from about 1 g to about 10 g, preferably from about 1 g to about 5 g.

This method for shaving with a manual razor comprises the steps of:

(a) wetting the area with water,

(b) applying an effective amount of the shave gel composition to the area

(c) working the shave gel composition into a generous lather with hands or shaving brush.

(d) shave with manual razor.

This method for shaving with electric clippers comprises the steps of:

(a) wetting the area with water,

(b) applying an effective amount of the shave gel composition to the area

(c) working the shave gel composition into a generous lather with hands

(d) rinse with water; pat dry, and

(e) shave with electric clippers

### EXAMPLES

The compositions illustrated in Examples I and II illustrate specific embodiments of the shave gel compositions of the present invention, but are not intended to be limiting thereof. Other modifications can be undertaken by the skilled artisan without departing from the spirit and scope of this invention. The compositions illustrated in Examples I and II are prepared in the following manner (all percentages are based on weight unless otherwise specified).

| | | |
|---|---|---|
| PHASE A | WATER (AQUA) | 41.20 |
| PHASE A | DISODIUM EDTA | 0.20 |
| PHASE A | PEG-14M | 1.00 |
| PHASE A | SODIUM CAPROYL LACTYLATE | 1.00 |
| PHASE A | PEG-150 DISTEARATE | 0.50 |
| PHASE A | BUTYLENE GLYCOL | 2.50 |
| PHASE A | PEG-8 DIMETHICONE PEG-8 RICINOLEATE | 0.50 |
| | POLYQUATERNIUM-57 | |
| PHASE B | COCAMIDOPROPYL BETAINE | 8.00 |
| PHASE B | SODIUM LAURETH SULFATE | 15.00 |
| PHASE B | SODIUM C14–16 OLEFIN SULFONATE | 40.00 |
| PHASE B | COCAMIDE MEA | 1.50 |
| PHASE C | FRAGRANCE | 0.15 |
| PHASE D | METHYLCHLOROISOTHIAZOLINONE | 0.05 |
| | METHYLISOTHIAZOLINONE | |
| PHASE D | GLYCOL DISTEARATE SODIUM LAURETH | 2.50 |
| | SULFATE COCAMIDE MEA LAURETH-7 | |
| PHASE D | DICETYLDIMONIUM CHLORIDE | 0.75 |

MANUFACTURING INSTRUCTIONS:

| | |
|---|---|
| PHASE A | COMBINE WATER AND DISODIUM EDTA AND |
| | MIX UNTIL UNIFORM. DISPERSE POLYOX INTO |
| | WATER AND MIX HEAT TO 80 C. AND ADD |
| | REMAINING PHASE A INGREDIENTS |
| PHASE B | ADD PHASE B INGREDIENTS TO BATCH SLOWLY |
| | COOL BATCH TO 30 C. |
| PHASE C | ADD PHASE C TO BATCH WITH MIXING |
| PHASE D | ADD PHASE D INGREDIENTS ONE AT A TIME |
| | WITH MIXING. |

Preferred viscosities range from about 4500 to about 12,000 centistokes at 25.degree. C. (as measured by a Brookfield RV4@20 RPM).

The compositions illustrated in Examples I-II all of which are embodiments of the present invention, provide excellent lubricity and conditioning of hair, and further enhance conditioning impression by providing excellent spreading through hair and generate thick, dense lather.

| | | |
|---|---|---|
| PHASE A | WATER (AQUA) | 41.20 |
| PHASE A | DISODIUM EDTA | 0.20 |
| PHASE A | PEG-7M | 0.50 |
| PHASE A | SODIUM LAUROYL LACTYLATE | 0.50 |
| PHASE A | PEG-150 DISTEARATE | 1.50 |
| PHASE A | GLYCERIN | 0.25 |
| PHASE A | PEG-8 DIMETHICONE PEG-8 RICINOLEATE | 2.00 |
| | POLYQUATERNIUM-57 | |
| PHASE B | COCAMIDOPROPYL BETAINE | 3.00 |
| PHASE B | SODIUM LAURETH SULFATE | 10.00 |
| PHASE B | SODIUM C14–16 OLEFIN SULFONATE | 20.00 |
| PHASE B | COCAMIDE MEA | 2.00 |
| PHASE C | FRAGRANCE | 0.30 |
| PHASE D | METHYLCHLOROISOTHIAZOLINONE | 0.05 |
| | METHYLISOTHIAZOLINONE | |
| PHASE D | GLYCOL DISTEARATE SODIUM LAURETH | 1.50 |
| | SULFATE COCAMIDE MEA LAURETH-7 | |
| PHASE D | DICETYLDIMONIUM CHLORIDE | 0.50 |

Preferred viscosities range from about 4500 to about 12,000 centistokes at 25.degree. C. (as measured by a Brookfield RV4@20 RPM).

What is claimed:

1. A shaving composition that provides slip and emolliency for manual razors and when rinsed off conditions the hair and allows for a closer shave with electric shaving devices.
(a) from about 5% to about 60% by weight of a detersive surfactant selected from the group consisting of anionic surfactant, nonionic surfactant, amphoteric surfactant, zwitterionic surfactant, and mixtures thereof;
(b) from about 0.05% to about 10% by weight of an ethoxylated silicone conditioning agent;
(c) from about 0.05% to about 5% by weight of dispersed droplets of a water insoluble, hair conditioning agent having a number average droplet diameter of from about 4.0 microns to about 500 microns;
(d) from about 0.1% to about 5% by weight of ethylene glycol distearate;
(e) from about 0.2% to about 6% by weight of sodium caproyl lactylate,
(f) from about 0.025% to about 1.5% by weight of polyalkylene glycol corresponding to the formula: ##STR15## wherein R is selected from the group consisting of hydrogen, methyl and mixtures thereof, and n is an integer having an average value of from about 1,500 to about 25,000; and
(g) from about 0.025% to about 5% by weight of an organic, cationic, non crosslinked, deposition polymer having a cationic charge density of from 4 meq/gm to about 7 meq/gm and an average molecular weight of from about 1,000 to about 500,000; and
(h) from about 20% to about 94.8% by weight of water.

2. The composition of claim 1 wherein R is hydrogen and n is an integer having an average value of from about 2,500 to about 20,000.

3. The composition of claim 2 wherein the composition comprises from about 0.1% to about 0.5% by weight of the polyalkylene glycol.

4. The composition of claim 1 wherein the silicone conditioning agent is ethoxylated dimethicone where the average degree of ethoxylation range from 2 to about 40 at a concentration of from about 0.2% to about 3% by weight of the composition.

5. The composition of claim 1 wherein said composition comprises from about 5% to about 30% by weight of anionic surfactant.

6. The composition of claim 1 wherein the composition further comprises a cationic conditioning agent.

7. The composition of claim 6 wherein the conditioning agent is a water soluble quaternary cellulose derivative at a concentration of from about 0.05% to about 3% by weight of the composition.

8. The composition of claim 1 wherein the sodium caproyl lactylate can be substituted with or used in combination with sodium lauroyl lactylate, sodium isostearyl lactylate, and/or isostearyl lactylate.

9. The composition of claims 1 wherein the use is personal shaving with a razor blade or electric shaving devices wherein said composition comprises materials selected from the group consisting of, rheology modifiers, conditioning agents, wetting agents, cleansing agents, lather producing compositions, and emollients (hydrophilic, lipophilic and silicone/silicone derived) individually or mixtures thereof.
